(19) United States
(12) Patent Application Publication
(10) Pub. No.: US 2024/0389846 A1
(43) Pub. Date: Nov. 28, 2024

(54) Methods for Localization of Cancerous Tissue Using Fluorescent Molecular Imaging Agent for Diagnosis or Treatment

(71) Applicant: Vergent Bioscience, Inc., Minneapolis, MN (US)

(72) Inventors: John T. Santini, JR., Chanhassen, MN (US); Eric Scott Bensen, Minneapolis, MN (US); Andrea Marie Simpson, Golden Valley, MN (US)

(21) Appl. No.: 18/793,215

(22) Filed: Aug. 2, 2024

Related U.S. Application Data
(63) Continuation-in-part of application No. PCT/US23/12210, filed on Feb. 2, 2023.
(60) Provisional application No. 63/306,019, filed on Feb. 2, 2022.

(51) Int. Cl. A61B 1/267; A61B 1/04; A61B 5/00; A61B 10/04

(52) U.S. Cl. CPC A61B 1/2676; A61B 1/043; A61B 1/046; A61B 5/0071; A61B 10/04; A61B 2010/045

(57) ABSTRACT

Methods are provided that include navigating an instrument, via a minimally invasive route (e.g., an endoluminal procedure), into a patient to whom a molecular imaging agent has been intravenously administered, to position the instrument in an area of a tissue abnormality; and visualizing, via the instrument, tissue in the area under near-infrared (NIR) light, wherein the molecular imaging agent, as administered, causes abnormal tissue in the area to fluoresce under the NIR light and enable the fluorescing abnormal tissue to be localized within the area. The method may further include diagnosing and/or treating the fluorescing abnormal tissue.

### CROSS-REFERENCE TO RELATED APPLICATIONS

This application is continuation-in-part of PCT/US2023/012210, filed Feb. 2, 2023, which claims priority to U.S. Provisional Patent Application No. 63/306,019, filed on Feb. 2, 2022, which are incorporated herein by reference.

### BACKGROUND

Lung cancer is the leading cause of cancer death, and the second most diagnosed cancer, in the United States and globally. Lung cancer diagnoses are expected to increase because the U.S. Preventative Services Task Force revised the lung cancer screening guidelines in early 2021 such that the number of people who are advised to undergo regular screening for lung cancer has nearly doubled. Today, most lung cancer is found in late stages where treatment options are limited, but advancements in lung cancer technology and clinical practice are increasing awareness of the disease and are expected to drive a lung cancer stage shift. When found early, lung cancer is highly treatable through surgery, so the stage shift is anticipated to dramatically and positively impact lung cancer outcomes, while also increasing the number of patients who are surgically eligible.

Historically, lung biopsies and resections were performed using an open thoracotomy procedure, but these procedures are becoming less common. Minimally invasive approaches to both biopsy and resection are becoming the standard of care across all surgical fields, and Minimally Invasive Surgery (MIS) reduces the risk of complications and trauma to the patient's body, thereby decreasing recovery times. When performing these MIS procedures, which include video-assisted thorascopic surgery (VATS), robotic surgery or robotic-assisted thorascopic surgery (RATS), surgeons may not be able to rely on palpation to help identify tumorous lung tissue because the holes used for these minimally invasive procedures may not be large enough for the surgeon to easily put their hands or fingers inside the patient. Instead, surgeons may need to more heavily rely on information gathered from preoperative scans while attempting to locate tumors using a miniature camera placed in the chest cavity or bronchial passages.

Lung imaging, such as chest CT (computed tomography) and x-ray, can identify lung nodules and, in conjunction with other patient information, is used to determine appropriate follow-up steps. For those nodules requiring action, physicians utilize a number of techniques to confirm if the nodule is malignant or benign. When diagnosing lung cancer, physicians identify potentially cancerous tumors with conventional screening and imaging techniques such as CT, positron emission tomography (PET)/CT, cone-beam CT, magnetic resonance imaging (MRI), x-ray, fluoroscopy, and ultrasound, among others. While these non-invasive imaging techniques have improved significantly in recent years, they are still not entirely effective in identifying and locating all potential lesions, nodules, tumors, or masses, whether benign or malignant. Moreover, these scans at best can only identify whether a patient has a nodule or tumor, but cannot provide information whether the tumor is cancerous. Confirming that a tumor is cancerous requires a biopsy. While it is possible that a lesion will be benign, it is still important to get a confirmatory diagnosis through both lesion biopsy and lymph node staging. Evaluating lesions that require follow-up quickly and accurately is important because lung cancer treatment is more successful when it is found and treated early.

The term “lesion” as used herein as a shorthand to broadly refer to any lesions, nodules, tumors, or masses, or the like, whether benign or malignant, for which a physician may want to localize, unless a specific one of these is expressly indicated. Accordingly, the terms “lesion”, “nodule” and “tumor” may be used interchangeably unless the context conveys that a particular term is intended.

Biopsies are usually performed endoscopically or transthoracically by interventional pulmonologists, thoracic surgeons, or interventional radiologists. Surgical wedge resections are also done by thoracic surgeons in cases where lung cancer probability is extremely high and/or when other methods have failed to reach a definitive diagnosis, but the lesion is highly suspicious for cancer. Lung cancer lymph node staging is typically performed by an interventional pulmonologist using linear-endobronchial ultrasound (EBUS) but may also be performed by a thoracic surgeon who does a surgical mediastinoscopy. While EBUS is widely adopted for staging and holds top clinical guideline position, there is mounting evidence that many patients are either mis-staged due to clinician error, lack of adequate tissue, or simply because the time from procedure to surgery was too long.

Despite many advancements in biopsy and staging from technology to blood biomarker testing and pathology techniques, the average time from abnormal finding or first symptom to treatment for lung cancer ranges from 3-6 months in the United States and globally. Challenges in the patient care continuum are a top focus area in many hospitals, yet confidently getting a diagnosis continues to challenge clinicians. Advanced bronchoscopy systems such as the Medtronic superDimension™ System and ILLUMISITE™ Platform, Olympus Veran SPIN System™, Bodyvision Lung Vision, Intuitive Ion™, Johnson and Johnson Auris Monarch™ Platform, Noah Medical Galaxy System™, and Bronchus Archimedes™ Systems all seek to improve biopsy through enabling minimally invasive access to all segments of the lung to collect tissue.

While these systems offer great advancement, challenges still exist in biopsy. Nodule localization with these approaches or in combination with enabling technologies such as fluoroscopy, cone-beam computed tomography systems (CBCT), radial endobronchial ultrasound (REBUS) and/or confocal laser endomicroscopy is challenging. Many technologies use pre-procedure CTs and virtual lesion location making real-time confirmation and localization difficult. Working channels may also be small, such that biopsy tools may only take small pieces of tissue challenging traditional pathology requirements and approaches.

Furthermore, even in cases where the nodule is located with confidence, only a part of the nodule may be malignant. As a result, physicians use multiple technologies and biopsy tools in order to take as much tissue as possible from around the area of interest in order to maximize the likelihood of getting a definitive answer. Some physicians bring cytotechnologists into the procedure room to further evaluate tissue adequacy through rapid onsite evaluation (ROSE). Others are investing in digital pathology systems used bedside with the goal of improving diagnostic yield and reducing procedure time. Diagnostic yield from advanced bronchoscopy continues to improve, yet there is significant unmet need in lesion identification, localization, and confirmation of diagnosis within the procedure.

Lung cancer diagnosis and staging is followed by surgical resection in cases where the patient is confirmed to have cancer and surgically eligible as determined by the stage of disease and the patient condition. Preoperative scans can provide the surgeon with the relative position of the nodule within the lung; however, these images are not well-translated to what the surgeon sees during the actual procedure. For example, preoperative scans are taken while the patient's lungs are fully inflated but in surgery the lungs are deflated. As a result, even the most experienced surgeons may encounter difficulties finding a suspected tumor during these procedures, often caused by the relatively small size (less than 2 cm) of many operable lung nodules, the continuous movement and flexibility of lung tissue, and the variability in the appearance of the tumors and healthy lung tissue.

These procedures therefore present a unique set of challenges for surgeons, especially when operating on tumors that are particularly small or difficult to locate. As a result, a surgeon may convert to, or begin with, a more complicated and aggressive open procedure to ensure they can confidently locate and remove the entirety of the tumor. These procedures are significantly more expensive and will often have increased recovery times. However, these disadvantages are far preferable to a “futile”—or unsuccessful—surgery, where the surgeon is unable to find or fully remove a tumor.

Especially for early-stage lesions, many surgeons favor tissue sparing resections to preserve lung function and enable possible repeat resections in patients with synchronous or metachronous lesions. Lung-sparing surgery is also central to the treatment of multifocal lung cancers, particularly in patients with limited pulmonary reserve. Unsuccessful localization of a pulmonary nodule during VATS is the most common reason for conversion to a full thoracotomy. The inability to identify the nodule targeted for resection increases significantly if the nodule is smaller than 10 mm or is located more than 5 mm from the pleural surface. Furthermore, during robotically assisted surgery, no incision is made to enable direct palpation. Various preoperative localization techniques have been developed to aid in intraoperative nodule identification during VATS and robotically assisted surgery. These localization techniques are intended to mark the nodule for guidance in resection and to provide some evidence of oncologic margin after resection.

Some surgeons will opt for a complicated preoperative marking procedure when they believe the tumor will be difficult to locate, or if the tumor is not necessarily confined to a predictable area of the lung. This preoperative marking procedure involves placing a fiducial marker (like a small metal coil or similar object) or injecting a radioactive dye (such as ^{99m}Tc) near the site of the tumor, either bronchoscopically by an interventional pulmonologist or percutaneously by an interventional radiologist. Another approach is transthoracic hook wire placement by an interventional radiologist. In some cases, the surgeon may perform the entire preoperative marking and surgical procedure. Of these methods, percutaneous injection seems to provide more accurate results, but it requires a hybrid operating room equipped with CT scanners or a separate interventional radiology procedure prior to surgery.

In yet another alternative procedure, surgeons can map the preoperative scans to the chest anatomy and bronchoscopically mark near the tumor with indocyanine green (ICG), methylene blue, or omnipaque. This procedure can be executed with advanced bronchoscopy systems such as the Medtronic superDimension™ System and ILLUMISITE™ Platform, Olympus Veran SPIN System™, Bodyvision Lung Vision, Intuitive Ion™, Johnson and Johnson Auris Monarch™ Platform, Noah Medical Galaxy System™, and Bronchus Archimedes™ or in some cases with a traditional or thin bronchoscope. While these procedures can provide strong visual guidance for the surgeon, there is also a risk that the any dye will not be accurately placed at the nodule, or that the dye will spread throughout the patient's tissue which defeats the purpose of the procedure.

To improve the accuracy of dye placement, surgeons may also utilize fixed or mobile CBCT imaging systems, such as those produced by Philips, Siemens, G E, and Zichm, which attempt to provide real-time navigational guidance by taking CT images to track the head of the endoscope with respect to the position of the nodule. These systems are particularly disadvantageous because the patient, physician, and procedural staff are repeatedly exposed to radiation, in addition to the fact that CBCT systems are very expensive and require a special, hybrid operating room to perform the procedure. Furthermore, the use of CBCT often significantly lengthens procedure time, resulting in longer anesthesia times and higher procedure costs. For example, most of these systems also require staff to leave the procedure room during CBCT spins, which may present workflow and procedural challenges. Whether CBCT is done in the same setting as the resection, or separately, CBCT may increase the accuracy of preoperative marking at the expense of adding complexity, time, and cost to the procedure.

In most preoperative marking procedures, the patient will require transportation between the interventional radiology or endoscopy suite and the operating room. In other cases, these may be done in a single procedure where the operating room has appropriate staff and technology. Regardless, timing and coordination of preoperative marking is complex and requires additional planning, equipment, procedure staff, and time. In cases where preoperative marking is performed, if the surgeon is still unable to locate the tumor and perform a MIS resection, the surgeon may later convert to a traditional open thoracotomy.

While surgery is the gold standard for early-stage operable patients, there is significant development in endoluminal therapies intended to address unmet clinical needs for those who are not able to have surgery. Endoluminal therapies include energy (microwave, radiofrequency, cryotherapy, vapor, pulsed electric field, and photodynamic therapy) and drug (chemotherapy, virus, or immunotherapy) delivered to locally treat the lesion. Local therapies, for example, may be delivered endoscopically through a bronchoscope or extended working channel by an interventional pulmonologist or thoracic surgeon, or percutaneously by an interventional radiologist. Advanced bronchoscopy systems are all anticipated to broadly enable local therapy delivery by pulmonologists and thoracic surgeons in the future. Interventional radiologists may offer some of these non-surgical therapies, but many are unwilling to take on the risk of complications associated with transthoracic lung treatment and the associated airway management.

Moreover, some surgeons, such as those in local, community hospitals, do not have access to the technology or skilled personnel required to perform these advanced marking procedures. Those surgeons will therefore perform a more aggressive resection or will convert to an open thoracotomy if they are unable to identify the location or boundaries of a tumor. Aggressive resections are not ideal as they can result in removal of excess amounts of healthy tissue, so converting to an open thoracotomy is generally preferable, even though such an open procedure still produces trauma that may result in longer hospital stays and recovery times for the patient.

Accordingly, there still remain many challenges and unmet clinical needs in lesion localization, diagnosis, and treatment. Thus, there is a significant need for more reliable, real-time intraoperative visualization techniques, for example, to better aid surgeons and pulmonologists in localizing and differentiating tumorous and healthy lung tissue when performing these procedures.

### BRIEF SUMMARY

The present disclosure provides improved methods for localizing, diagnosing, and treating cancer, including but not limited to methods that include using minimally invasive surgical procedures.

In one aspect, a method is provided that includes: navigating an instrument, via a minimally invasive route (e.g., an endoluminal procedure), into a patient to whom a molecular imaging agent has been intravenously administered, to position the instrument in an area of a tissue abnormality; and visualizing, via the instrument, tissue in the area under near-infrared (NIR) light, wherein the molecular imaging agent, as administered, causes abnormal tissue in the area to fluoresce under the NIR light and enable the fluorescing abnormal tissue to be localized within the area. The method may further include diagnosing and/or treating the fluorescing abnormal tissue.

In another aspect, a method is provided that includes navigating an instrument, via an endoluminal route, into a patient to whom VGT-309 (a particular molecular imaging agent, defined below) has been intravenously administered, to position the instrument in a target area; visualizing, via the instrument, the target area under near-infrared (NIR) light; and identifying in real time the location of any cancerous tissue within the target area by the florescence of the cancerous tissue caused by the VGT-309 under the NIR light.

These methods are particularly advantageous in localizing, diagnosing, and treating various cancer and solid tumors. For example, the methods may be used to endoscopically localize, diagnose, and treat lung cancer, as well as colorectal, gastric, and esophageal cancers.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may or may not be present in various embodiments. Elements and/or components are not necessarily drawn to scale.

FIGS. 1A and 1B depict a lesion (abnormal tissue) located outside of a distal airway of a patient.

FIG. 1C depicts the location of the lesion of FIG. 1A from a perspective within an airway, illustrating that it cannot be visualized through the airway wall.

FIG. 2A depicts a lesion located outside of a distal airway of a patient and “lighting up” following administration of a molecular imaging agent, according to an embodiment of the present disclosure.

FIG. 2B is a close-up view of the lesion of FIG. 2A, according to an embodiment of the present disclosure.

FIG. 2C depicts visualization of the lesion of FIG. 2A from within the airway, according to one embodiment of the present disclosure.

FIG. 2D depicts visualization, from within an airway, of a lesion outside of a distal airway of a patient with only part of the lesion “lighting up” following administration of a molecular imaging agent, according to one embodiment of the present disclosure.

FIG. 3A depicts a method of biopsying a lesion (abnormal tissue) located outside of a distal airway of a patient following administration of a molecular imaging agent to light up the lesion, according to one embodiment of the present disclosure.

FIG. 3B depicts the method of FIG. 3A viewed from within the airway, according to an embodiment of the present disclosure.

FIG. 3C depicts a method of visualization and biopsying of a lesion located outside of a distal airway of a patient, viewed from within the airway, with only part of the lesion “lighting up” following administration of a molecular imaging agent to the patient, according to an embodiment of the present disclosure.

FIG. 4A depicts a method of visualization and treatment of a lesion located outside of the distal airway of a patient following administration of a molecular imaging agent, wherein the lesion is “lit up” to guide a treatment instrument into/through the lesion, according to an embodiment of the present disclosure.

FIG. 4B depicts the method of FIG. 4A viewed from within the airway, according to an embodiment of the present disclosure.

FIG. 4C depicts a method of visualization and treatment of a lesion located outside of a distal airway of a patient, viewed from within the airway, with only part of the lesion “lighting up” following administration of a molecular imaging agent to the patient, wherein a treatment instrument is guided into/through the “lit up” part of lesion according to one embodiment of the present disclosure.

FIGS. 5A-5D are illustrations of tissue abnormalities commonly attributable to colorectal cancer.

As used throughout the figures, shading represents tissue that is fluorescent when viewed under near-infrared (NIR) light after a molecular imaging agent has been administered to a patient, as described herein.

### DETAILED DESCRIPTION

Methods are described for using fluorescent molecular imaging agents, particularly near-infrared (NIR) fluorescent imaging agents, to localize tissue abnormalities in a patient for diagnosis and/or treatment, particularly in real-time during a medical procedure, such as a minimally invasive procedure, e.g., an endoscopic procedure. The diagnosis and/or treatments include but are not limited to biopsies, resection or ablation of, or drug delivery to, a localized tumor.

In some particular embodiments, the localization methods include (i) navigating an instrument, via an endoluminal route or other minimally invasive route, into a patient to whom a molecular imaging agent, such as VGT-309 (defined below) has been intravenously administered, to position the instrument in a target area, e.g., an area in which a tissue abnormality is believed to be located; (ii) visualizing, via the instrument, the target area under near-infrared (NIR) light; and (iii) identifying in real time the location of abnormal tissue within the target area by the florescence of the abnormal tissue caused by the molecular imaging agent under the NIR light. The methods typically would further include diagnosing and/or treating the localized tissue, which may entail further positioning a portion of an instrument inside (or near, if sufficient) the lit up portion to take a biopsy or deliver a therapy. Advantageously, the fluorescing tissue provides a fixed, visual reference point (like a north star) for instrument guidance, particularly in an endoluminal procedure where the distal end of instrument is advanced/steered based, at least in part, on tissue landscape viewed from the distal end of the instrument (as opposed, for example, to a virtual reference point used by navigational software).

The instruments using in this method include conventional scopes (e.g., endoscope, bronchoscope, thorascope, laparoscope, etc.,) including cameras and light sources, as well as catheters, biopsy needles, and other conventional tools for tissue acquisition or diagnosis and delivery of therapeutics and tools for ablation or local treatment by any means known in the art.

NIR light is generally understood in the art as having a wavelength in the range of 700 to 900 nm. As used herein, reference to “visualizing” tissue “under near-infrared (NIR) light” and causing tissue “to fluoresce under the NIR light” refer to a process of directing NIR light (e.g., having a wavelength in the range of 770 to 810 nm) at tissue and receiving/sensing the NIR emissions (e.g., having a wavelength in the range of 790 to 850 nm) generated in response. The received emissions are manipulated as known in the art to enable a physician to see the fluorescing of the tissue through a scope deployed near the tissue. Advantages of using NIR light include that it (i) has effective tissue penetration (e.g., at least 5 to 10 mm), (ii) has limited interference by biological tissue, because blood absorbs below 650 nm and water/lipids absorb above 900 nm, and (iii) does not generate significant autofluorescence. In a preferred embodiment of the methods described the NIR light wavelengths are selected for optimal imaging using ICG or ICG-containing molecular imaging agents.

These methods may be used to localize nodules as a standalone or combination modality in conventional biopsy and therapy conducted through endoluminal approaches. These methods may be used to identify and localize lesion hot spots, i.e., those parts of the lesion that are cancerous, as not all of the lesion will be cancerous in every case. These methods may be used in lymph node identification, for example, as confirmation in conjunction with another diagnostic procedure, e.g., before an endobronchial ultrasound bronchoscopy. These methods may be used in a lymph node evaluation or assessment without tissue acquisition. These methods may be used to facilitate biopsy, staging, and an appropriate local treatment of cancerous tissue within a single procedure. These localization methods may be used to facilitate quick and accurate catheter placement in a lesion for local therapy delivery and to enhance understanding of ablation treatment zones/nodule destruction. In various embodiments, these localization methods may replace, or may augment, conventional approaches for confirming the presence of cancer cells, including but not limited to white light, fluoroscopy, CBCT, radial endobronchial ultrasound (rEBUS).

One benefit of the present methods is that it can provide physicians with increased confidence in biopsy results, because the physician's increased certainty that they are acquiring tissue from the fluorescing portion of a nodule. They can share this additional evidence with the patient and a tumor board to create greater confidence in the diagnostic outcome, which may shorten treatment time and/or reduce unnecessary additional procedures.

The diagnostic methods described herein may decrease the need for a surgeon's reliance on conventional rapid diagnostic methods such as frozen section pathology or rapid on-site evaluation (ROSE).

As used herein the term “patient” refers generally to humans, but the methods described herein could be applied to other mammals, for example in pre-clinical animal models or veterinary applications.

The molecular imaging agent is one that covalently binds to a target molecule (such as a cathepsin, which is a type of protease) that is present at increased levels in solid tumors, tumor-associated macrophages (TAMS), and the tumor microenvironment. In a particular embodiment, the molecular imaging agent includes indocyanine green (ICG) as the NIR dye and can be used with a variety of conventional, commercial imaging systems. In some embodiments, the molecular imaging agent is a covalent, activatable, protease targeted imaging molecule, such as described in U.S. Pat. No. 10,100,037 to Bogyo et al., which is incorporated herein by reference. In some embodiments, the molecular imaging agent is one that includes ICG as the fluorophore, such as described in U.S. Pat. No. 10,869,936 to Bogyo et al., which is incorporated herein by reference.

In a preferred embodiment of the methods described herein, the molecular imaging agent is referred to herein as “VGT-309”, which comprises:

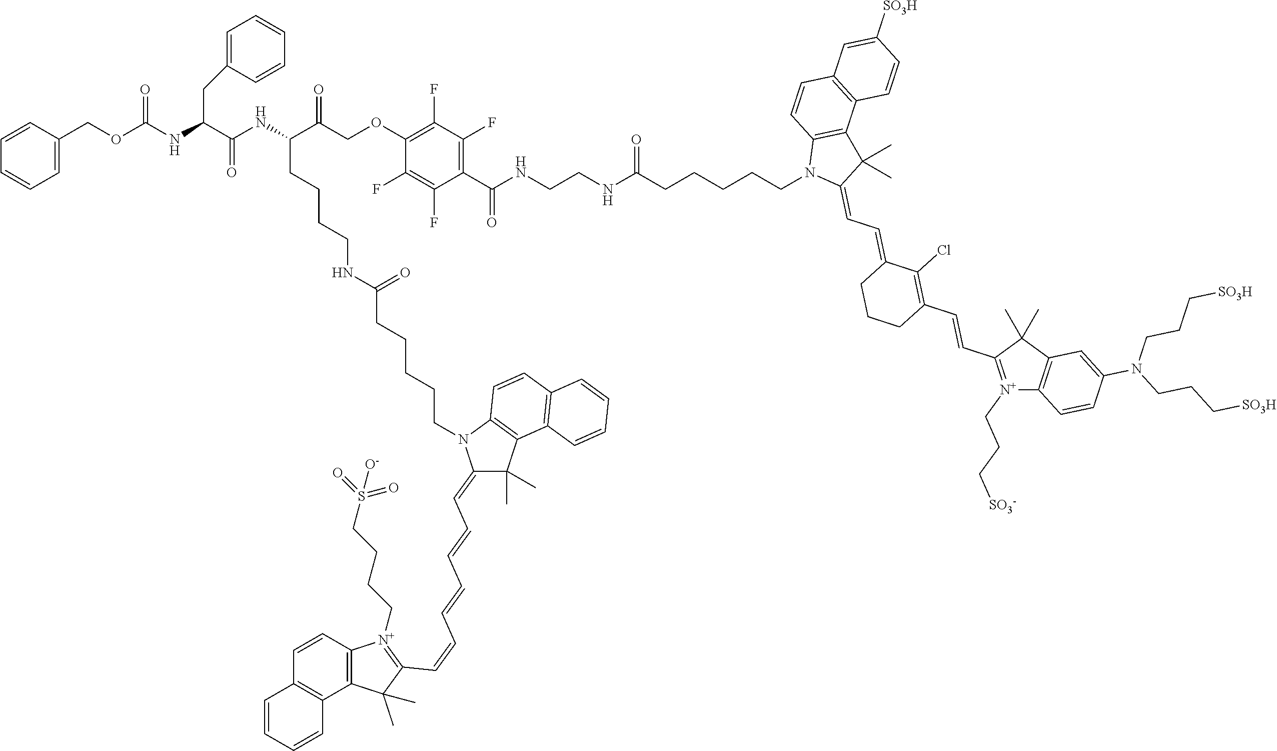

and includes pharmaceutically acceptable salts or formulation variants thereof. VGT-309 advantageously covalently binds to its target and remains at the target site in a wide variety of tumor types.

In a preferred embodiment of the methods described herein, the molecular imaging agent is a sodium salt form of VGT-309.

In certain embodiments, these methods improve diagnostic procedures and the efficacy of surgical tumor resection and removal. While the present disclosure describes applications in the localization, diagnosis, and treatment of lung cancer, a person of skill in the art would recognize that these teachings can be applied to the diagnosis and treatment of other forms of cancer. For example, the disclosures herein can be applied to other cancers, especially, among others, colorectal, gastric, and esophageal cancers.

In particular, as VGT-309 targets cathepsins, which are present in larger amounts in solid tumors than in normal tissues. VGT-309 may be used with a variety of cancers, including brain, breast, colorectal, esophageal, gastric, liver, lung, melanoma, ovarian, pancreatic, prostate, and thyroid, where solid tumors have been shown to have cathepsins present at increased levels over normal tissue. Accordingly, the present methods can be applied to localize abnormal tissue at variety of locations with a patient's body.

In certain embodiments, VGT-309 may be administered to human patients at dosages from 0.01 mg/kg to 0.7 mg/kg, for example from 0.016 mg/kg to 0.64 mg/kg. In some preferred embodiments, the dosage is from 0.3 to 0.4 mg/kg, such as 0.32 mg/kg, administered from 2 hours to 36 hours before a medical procedure, involving NIR imaging of the patient's tissue as described herein, is untaken. In some embodiments, the dosage is 0.16 mg/kg, 12 to 36 hours prior to the procedure. In some embodiments, the dosage is 0.32 mg/kg, from 2 to 6 hours, or from 12 to 36 hours, prior to the procedure. In some embodiments, the dosage may be from 0.32 to 0.52 mg/kg, such as 0.5 mg/kg, from 2 hours to 48 hours prior to the procedure. In some other embodiments, the dosage may be up to 0.64 mg/kg, for example from 2 hours to 48 hours prior to the procedure.

### Localization

In recent years there has been a shift towards minimally invasive surgical procedures which, unlike open thoracotomies, are performed through a few small holes and therefore limit the surgeon's vision and/or sense of touch. As used herein, “surgeon” refers to a physician capable of performing any of the procedures described herein, such as a cardiothoracic surgeon, a surgical oncologist, or interventional pulmonologist. During these procedures, surgeons are often concerned with their ability to find and localize known lesions, ensure adequate resection margins, and identify primary lesions, synchronous and metachronous lesions, as well as metastatic tumor spread, not identified by preoperative scans.

There has also been a shift to less invasive advanced bronchoscopy procedures for biopsy which are limited by physician skill, system inaccuracy, the lack of real-time nodule visualization and localization, instrumentation and biopsy tool capability, and pathology. Surgeons performing these procedures are challenged by lack of ability to localize known lesions, biopsy these lesions, and confidently confirm whether they are benign or malignant.

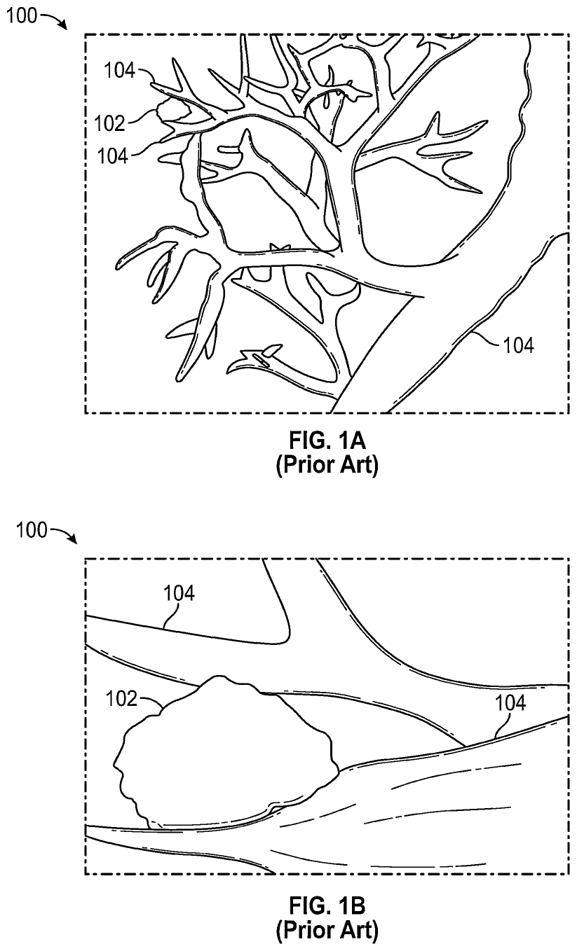

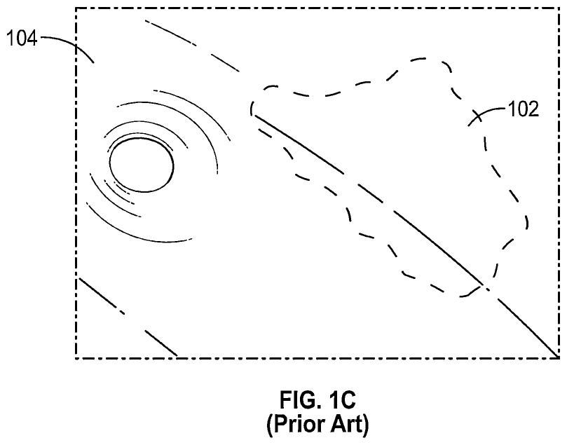

Physicians typically have limited information available to guide them during a biopsy, for example. Some technologies use multiple CT or MRI scans and software to create a three-dimensional (3D) model 100, an example of which is depicted in FIGS. 1A-1B. The physician may use such models 100 to guide an endoscope down the tortuous airway 104 toward a general area where the physician believes a lesion 102 is located. While these models may provide enough information for the physician to navigate a biopsy tool to the general location of a lesion 102, the physician may struggle to precisely locate and the lesions 102, since, as, the physician performing a bronchoscopy may be unable to see (using visible light) the lesion when viewing from within the airway 104, particularly because lesions 102 are more often than not positioned just outside or in the walls of the airway 104, as depicted in FIG. 1C. For example, the physician may see a general outline indicating a tissue abnormality, but there is no way for the physician to be certain that this is even a lesion 102, let alone the particular lesion 102 the physician is attempting to locate.

The presently disclosed methods, however, utilize a molecular imaging agent to “light up” the abnormal tissue (nodule, mass, tumor) or a portion thereof (for example, in a heterogenous lesion) thereby enabling the physician to localize and align an instrument (e.g., biopsy needle), seeing it in real-time, and apply the instrument to the abnormal tissue. The abnormal tissue that lights up is likely to be malignant (cancerous) but it is possible that abnormal tissue that lights up is benign. In either case, the lighting up and localization of the abnormal tissue is a useful outcome for the surgeon, as it may help the surgeon avoid converting to an open surgery.

Molecular imaging agents have the potential to significantly improve patient outcomes when used during biopsy and surgical removal procedures, which often are MIS procedures. Surgeons are relying solely upon white light visualization or virtual navigation with various external imaging modalities to locate and identify tumors during these procedures, which can often be challenging given the limited field of view in the small spaces of the body and the complexity of combining multiple modalities within a single procedure. Preoperative marking can aid surgeons in nodule localization and margin evaluation, but is limited by inaccurate advanced bronchoscopy systems, dye spread throughout the lung in cases where dye is utilized, and operational, skill, and scheduling challenges in various hospital settings. And even during open thoracotomies, where palpation may be used to identify nodules, surgeons may still struggle to locate and identify these nodules when they are small or obscured by the anatomy of the lungs. Thus, there is a significant need in the art for fluorescent molecules that target tumor cells, or the surrounding extracellular matrix, to aid surgeons in locating nodules identified on preoperative scans in both biopsy and treatment.

There are several molecular imaging agents that are currently in various stages of development and testing, but of these agents VGT-309 is particularly advantageous. VGT-309 targets and covalently binds to cathepsin targets, which are present in larger amounts in solid tumors and tumor margins than in normal tissues. When this binding occurs, the quencher is cleaved from the molecule and VGT-309 becomes “active” at which point the tumor will fluoresce under near-infrared (NIR) light. Additionally, based on preclinical studies, the covalent bond creates a wide therapeutic window such that VGT-309 can fluoresce cancerous tissue anywhere from two hours to four days after VGT-309 is administered. (Animal model studies also suggest that it may be possible for VGT-309 to continue to emit a signal in tissue out to seven days following administration.) This extended therapeutic window may be particularly beneficial in cases where the biopsy and resection procedures cannot be scheduled in a short time window, but only one administration of VGT-309 is required.

Although the present disclosure highlights how VGT-309 can make MIS procedures more efficient and effective, VGT-309 can also add value to standard open surgical procedures because VGT-309 can help the surgeon find the primary tumor quickly, define the boundaries of the tumor when deciding where to cut, and can discover additional small primary and metastatic tumors that were not able to be seen using preoperative scans or by the surgeon during the procedure just using their eyes and hands.

Another advantage of VGT-309 is that, particularly with respect to the lung surgery market, laparoscopic and robotic near-infrared (NIR) fluorescent imaging systems are already readily available in hospitals. Some of these NIR imaging systems include those developed by Intuitive Surgical, Olympus, Medtronic, Johnson & Johnson, Bracco, and Stryker. While flexible manual bronchoscopy systems (such as those from Olympus, Fujinon, Pentax, and Storz as well as advanced systems such as Medtronic superDimension™ System and ILLUMISITE™ Platform, Olympus Veran SPIN System™, Bodyvision Lung Vision and Bronchus Archimedes™ System) and flexible robotic bronchoscopy devices (such as the Intuitive Ion™, Johnson and Johnson Auris Monarch™ Platform, and Noah Medical Galaxy System™), which are used to perform lung biopsies and, in the future, deliver local therapies, are not yet equipped with NIR capabilities, as use of molecular imaging agents develops with respect to lung surgery it is likely that the advanced bronchoscopy market will follow and NIR capability will be added to these systems.

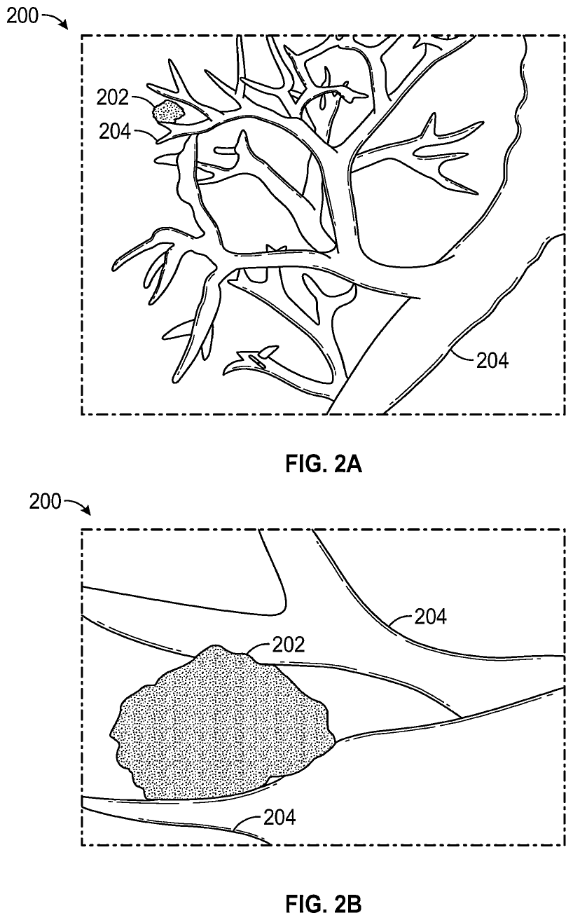

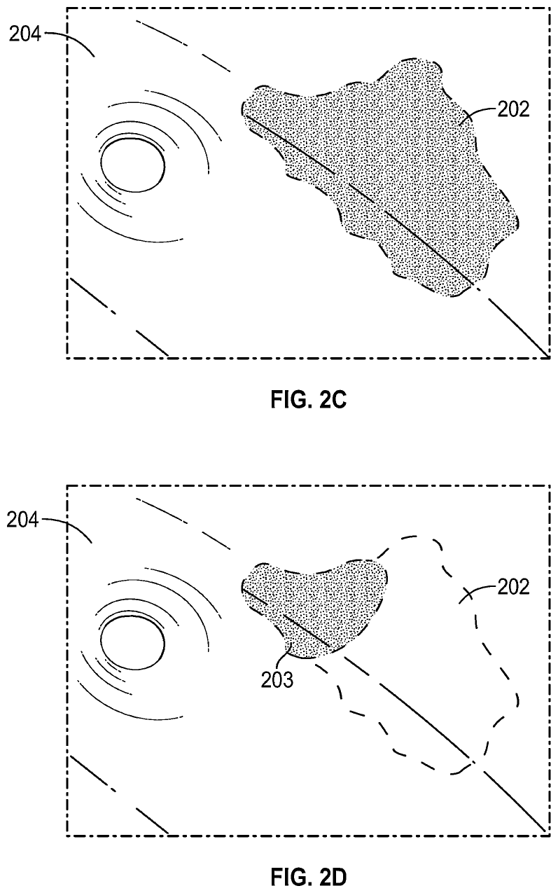

In use, VGT-309 may be intravenously administered prior to surgery or non-surgical local treatments to improve the surgeon's ability to visualize any tumors during MIS procedures. It has been demonstrated that a surgeon's visualization of tumors is improved when incorporating VGT-309 and NIR into a MIS procedure. Similar to the 3D models depicted in FIGS. 1A-1B, FIGS. 2A-2B depict 3D imaging models 200 of the airway 204 highlighting the illuminated tumor 202. Under white light, the tissue outside or in luminal wall of the airway 204 may seem abnormal, but it is not obvious where any tumors are located, if there are any tumors at all. Thus, in this case, a surgeon may take a more aggressive measure to identify and/or remove any abnormal tissue 202, which may involve an overly aggressive resection or an open thoracotomy. While both courses of treatment are more aggressive and improve the likelihood that all cancer is successful removed, there is still a risk that not all cancerous tissue will be identifiable.

In contrast, when using NIR to visualize lung tissue after the patient has been given VGT-309, the cancerous tissue 202 fluoresces, enabling the surgeon to clearly, and rather easily, identify what tissue is cancerous. As shown in FIG. 2C, an imaging device may be configured to display images of the airway 204 on a screen with the NIR fluorescence shown in a visible color, e.g., green, to help the surgeon see which tissue 202 is fluorescing. So instead of resecting all of the tissue in that area, or resecting only some of the tissue, the surgeon can more precisely identify and remove only the cancerous tissue 202. In this way, the surgeon can be more confident that they are resecting the tumors 202 with clean margins such that no cancerous tissue 202 is left behind.

It is also possible that a portion 203 of a lesion 202 may be more fluorescent than the rest of the lesion 202. In some cases, the highly fluorescent portion 203 may be representative of a higher concentration of cathepsins. This would indicate to the surgeon that the highly fluorescent portion 203 is a tumor “hot spot” (i.e., highly cancerous tissue). In other cases, as shown in FIG. 2D, only a portion 203 of the lesion may be fluorescent, which would indicate that only the fluorescent portion 203 of the lesion 202 is potentially cancerous. In either case, the surgeon can more accurately target the portion 203 of the lesion 202 that is cancerous, or more likely to be cancerous.

Accordingly, VGT-309 can provide surgeons with critical information during the diagnostic and treatment processes that they otherwise would not have access to, and as such may be particularly helpful in localizing, diagnosing, and treating lung cancer. When a lung nodule or abnormality is identified on a patient's scans, the next step for the patient will likely be a biopsy to confirm whether the nodule is cancerous. And by administering VGT-309 prior to biopsy, the pulmonologist and/or surgeon greatly increases the likelihood that the biopsy results will be accurate, thereby decreasing the chance that a patient will be incorrectly diagnosed or required to undergo additional or more invasive procedures in order to confirm the diagnosis.

The methods of localization described herein may be used with essentially any minimally invasive tools/equipment for accessing internal tissue target sites, whether conventional or developed in the future. For example, these tools/equipment may include advanced endoluminal approaches, such as catheter based robots.

### Diagnosis

Turning to the specifics of the diagnostic process, VGT-309 can be used in conjunction with traditional pathology to provide the pulmonologist with higher confidence in their diagnosis. Preoperative scans are often helpful in locating a tumor, but these scans cannot provide information as to whether the tumor is cancerous. Particularly in the context of lung cancer, proper diagnosis and staging is critical and it is almost entirely determinative of the remaining course of treatment. While a patient with a localized tumor, or early-stage cancer, requires a less aggressive course of treatment than a patient with metastatic spread in the later stages of cancer, it is nonetheless important to identify and treat the cancer early so that the patient has the highest possible likelihood of survival.

When performing a biopsy, a surgeon is guided by the limited information offered from the patient's preoperative scans. Typically, these scans will at best enable the surgeon to determine the general area of the nodule, but surgeons are still often faced with the difficult task of identifying the exact location of a nodule, often using virtual navigation in combination with a secondary imaging system, such as fluoroscopy, REBUS, CBCT, or confocal laser endomicroscopy. In systems where white light cameras are used, there is minimal utility in the periphery of the lung where the airways are very small, and lesions are typically outside a direct airway.

As previously described with respect to FIGS. 1A-IC, surgeons generally have limited resources available to guide them during a lung biopsy. Preoperative scans may be effective to generate a 3D model for guiding an endoscope within a patient's airway to the general location of a lesion, but a surgeon may still struggle to precisely locate and biopsy the lesion from within the airway. However, if the pulmonologist is using VGT-309 and NIR, they will have additional guidance when attempting to locate and biopsy the tumor. The fluorescence under NIR clearly distinguishes a malignant nodule from the healthy tissue, giving the pulmonologist a precise guide once they have successful navigated the biopsy needle to the general location of the tumor identified in the preoperative scans.

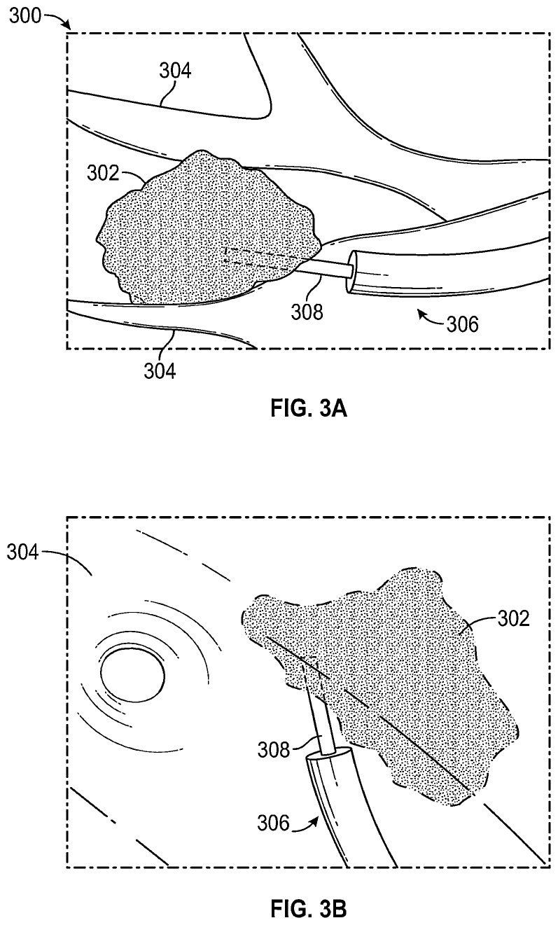

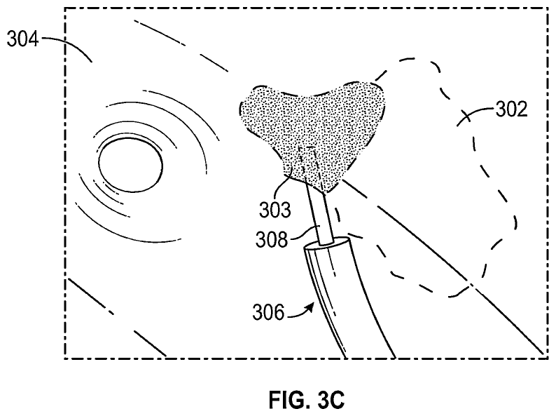

FIGS. 3A-3C illustrate the concept of VGT-309 lighting up the cancerous lesions 302, so that when an endoscope with NIR imaging capability is used, the surgeon can determine where to biopsy to get tissue that would be the most likely to give them an accurate assessment of whether cancer is present. Using VGT-309, surgeons can localize a lesion 302 in the lung near airway 304 in real time, align their biopsy tool or other instruments with the lesion 302, and accurately target and sample the lesion 302. That is, VGT-309 provides a direct guide for surgeons performing bronchoscopy procedures so that they may position their biopsy instrument appropriately to obtain the most accurate diagnosis possible. For example, a lesion may be illuminated to varying degrees, i.e., certain areas of the lesion may be more vibrantly illuminated than others. In these cases, the surgeon may target the portion 303 of the lesion 302 that is more vibrantly illuminated, as this portion 303 of the lesion 302 is most likely to be cancerous as compared to the rest of the lesion. Existing technologies do not provide such capabilities.

It is also possible that a patient has a nodule that is only partially cancerous, as shown in FIG. 3C, which may give rise to a situation wherein the patient is given the incorrect diagnosis because the surgeon was unable to identify or biopsy the specific cancerous portion of the nodule. VGT-309 and NIR can be particularly advantageous in these cases where only a portion 303 of the nodule 302 is cancerous, at least in that the surgeon can appropriately visualize the cancerous portion of the nodule to guide the biopsy. For example, if VGT-309 is administered to the patient, and then a robotic endoscope with NIR capability is used, then the surgeon will be able to see the illuminated region 303 of the nodule 302. That is, only the cancerous portion 303 of the nodule 302 will fluoresce under NIR. As compared to the overall size of the nodule 302, the cancerous portion 303 may be relatively small, so absent VGT-309 and NIR the surgeon would have a greater chance of collecting a biopsy from the non-cancerous portion of the nodule. That is, the VGT-309 enables the biopsy to accurately target the illuminated cancerous region 303 of the nodule 302.

In some cases, the surgeon may initially place the biopsy tool incorrectly, such that the biopsy tool is not reaching the cancerous portion of the nodule, which is fluorescing. In procedures where VGT-309 and NIR are not used, the surgeon may proceed to take the biopsy from this non-cancerous portion, because no conventional tools can give them any assessment of which part of a nodule might be cancerous and which may be benign, which may result in giving the patient and incorrect diagnosis. However, when the cancerous portion of the nodule is fluorescing the surgeon has an opportunity to correct the path of the biopsy tool to ensure the biopsy is taken from the correct portion of the nodule so that the diagnosis is as accurate as possible.

This has the further benefit of reducing the need for additional procedures, as it will increase physician confidence in the final pathology from the procedure. Without VGT-309 and NIR imaging, patients may be inaccurately diagnosed or may be required to undergo multiple, additional, and/or more invasive procedures.

VGT-309 may also aid in bronchoscopic lymph node evaluation. For example, it may eliminate the need for tissue biopsy as an independent diagnostic agent in lighting up cancerous lymph nodes. The ability to light up such lymph nodes may fully unlock the ability to biopsy, stage, and treat in a single procedure. Conventional lymph node biopsy and staging require histological pathology which takes multiple days to complete. VGT-309 may allow staging evaluation in a single diagnostic and treatment procedure where the patient is able to undergo potentially curative surgery or non-surgical endoluminal therapy during a single anesthetic event.

Physicians may also use VGT-309 as an independent diagnostic agent during surgery or non-surgical endoluminal treatment, such that intraoperative pathology reads—otherwise known as frozen sections—are no longer necessary. This would allow pulmonologists and/or surgeons to make decisions about what cancer is or is not cancerous in real-time, without having to wait for pathology results. VGT-309 can also eliminate the need for conventional pre-surgical marking techniques that some surgeons use to identify small nodules during resection procedures.

In some other embodiments, VGT-309 may be used in combination with other technologies, like those being developed by Mauna Kea Technologies, to further optimize the diagnostic process. For example, Mauna Kea has developed a series of in vivo cellular imaging technologies, including a catheter fitted with a NIR confocal microscope. This microscope may be guided to and inserted into a nodule, like performing a biopsy, and could detect low level of VGT-309 fluorescence within the cells themselves. Combining the information gathered by the fluorescent cell imaging with the information already provided with the Mauna Kea imaging platform could eliminate the need to perform biopsies altogether.

After a biopsy confirms the patient's nodule is cancerous, the next step in treatment is often a surgical resection. As previously discussed, these procedures often are performed using laparoscopic, endoscopic, or robotic techniques employing scalpels or thermal, ultrasonic, or laser cutters. When VGT-309 is administered prior to surgery, the surgeon will be able to better locate, identify, and resect tumors, while also inspecting the surrounding tissue to confirm the cancer has not spread to other parts of the patient's lungs.

### Methods of Treatment

VGT-309 and NIR may also be used in non-surgical tumor destruction procedures, like ablation, drug delivery, Pulsed Electric Field (PEF) or photodynamic therapy (PDT). Like the other procedures described herein, ablation and other non-surgical local therapies are minimally invasive but is advantageous as compared to surgical resection because they generally maximize preservation of healthy tissue. These non-surgical approaches will also expand the patient population able to undergo treatment, as they are typically safe for inoperable patient populations when delivered endoluminally, and because they may be used alone or in combination with other treatment modalities.

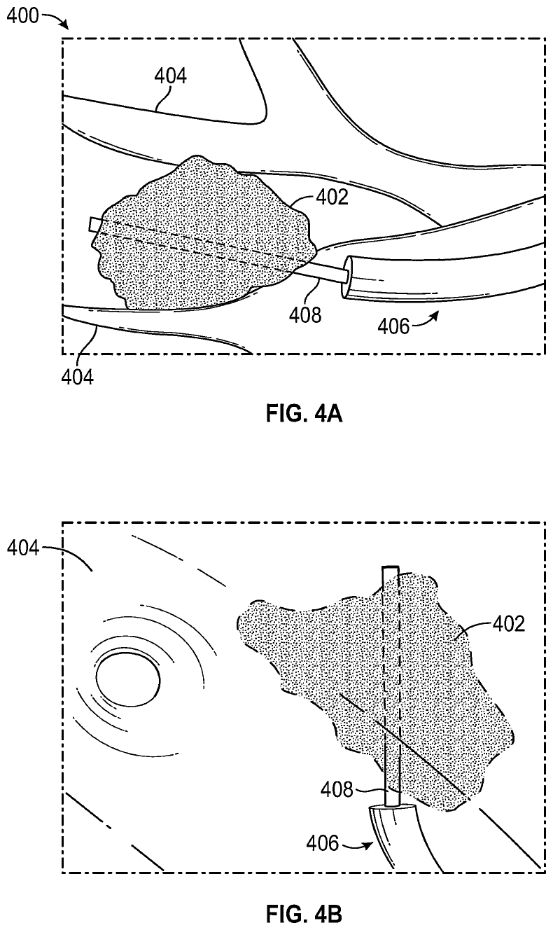

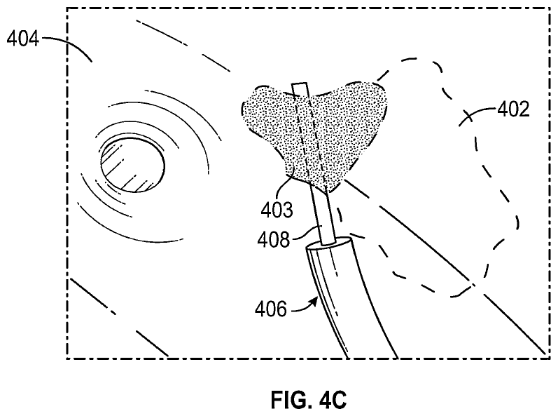

FIGS. 4A-4C illustrate the concept of VGT-309 lighting up the cancerous lesions 402 adjacent the airway 404, so that when an endoscope with NIR imaging capability is used, the surgeon can accurately administer a localized therapy to the lesion 402 via a treatment device. Surgeons must be very precise when performing ablation and other local therapy procedures, as the proximity of the treatment device (e.g., ablation catheter) to the tumor 402 (FIGS. 4A-4B), or a cancerous portion 403 of the lesion 402 (FIG. 4C), is critical in determining the efficacy of the procedure. Unlike biopsy, endoluminal ablation and other local therapies require that the delivery device completely traverse the lesion 402 to maximize the efficacy of the treatment and improve treatment margins. Because such precision is required in performing these ablation procedures, incorporating VGT-309 and NIR would be especially advantageous.

Like biopsy procedures, the ablation catheter may be navigated bronchoscopically to the general area of the tumor 402, but the same complications arise when pinpointing the exact location of the tumor 402. These procedures are clinically challenging, often require access to fixed CBCT systems, and are not yet widely available like those for performing other MIS procedures. These instrumentation systems that are in development do not include NIR capabilities, but incorporating NIR should not significantly alter the design or functionality of these systems.

### Alternative Applications

### Artificial Intelligence

Additionally, data collected during bronchoscopies and surgeries performed with VGT-309 and NIR may be used to train artificial intelligence (AI) algorithms that could be used to improve the efficacy of future surgeries. Specifically, an AI algorithm trained using images and videos of surgeries performed with VGT-309 can improve how these biopsies and/or resection procedures are performed, particularly as it pertains to identification and localization of cancerous tumors.

### Non-Lung Cancers

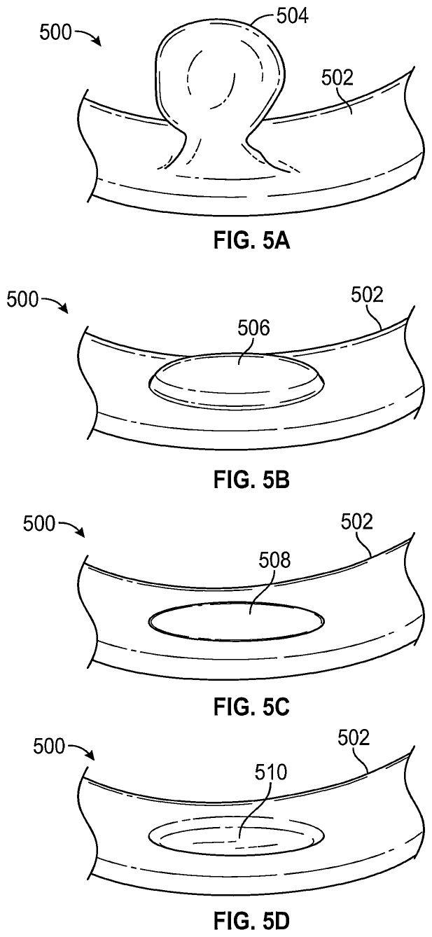

VGT-309 may also be particularly useful in diagnosing and treating colorectal cancer, although it is notable that colorectal cancer manifests differently than lung cancer. Unlike lung cancer, which manifests as tumors, colorectal cancer may be found in various forms, as shown in FIGS. 5A-5D. These may include polyps (FIG. 5A) and/or lesions, including elevated lesions (FIG. 5B), flat lesions (FIG. 5C), and depressed lesions (FIG. 5D).

Molecular imaging agents, including VGT-309, and NIR can be incorporated into conventional colonoscopy procedures to better aid surgeons in identifying cancerous polyps and/or lesions. Lesions, especially when they are flat or depressed, are difficult for surgeons to identify when the endoscope is being guided only by white light. While raised polyps may be more likely to be identified with only white light, flat or depressed lesions are more likely to blend in with the ordinary tissue of the colon. Because some of these lesions may be difficult to identify, there is a risk that surgeons will not identify any or all lesions and/or polyps present in a patient's colon, which consequentially may leave the patient with undiagnosed and/or untreated cancer. However, a surgeon using VGT-309 and NIR during routine colonoscopies will be more likely to identify otherwise hard to spot polyps and/or lesions, and therefore will be better able to correctly diagnose their patients' colorectal cancer, if necessary.

Moreover, because the standard of care for treating colorectal cancer is much different than that for treating lung cancer, VGT-309 and NIR are more likely to be applied during the diagnostic process than during resection and removal. When treating lung cancer, surgeons are highly concerned with preserving as much healthy tissue during the resection process. Colorectal cancer treatment is much more aggressive. Surgeons are less concerned with preserving healthy tissue when treating colorectal cancer, and therefore will remove polyps and/or lesions with large margins. However, it is possible that VGT-309 and NIR can still be used to confirm that, even with the aggressive resection, there are no residual traces of cancer in the patient's colon or nearby peritoneum.

Aside from resecting localized tumors with clean margins, surgeons are also concerned with ensuring that tissue surrounding the lungs, in particular the lymph nodes, have not been invaded by cancer. During these procedures, surgeons spend a significant amount of time taking biopsies of lymph nodes and sending those biopsies to pathology for frozen sections. Each frozen section can take anywhere from fifteen to thirty minutes, all while the patient remains under anesthesia. The prolonged length of this procedure can increase the patient's risk of surgical complications, while also increasing the overall cost of the surgery. However, surgeons also want to be sure that they have properly diagnosed and staged their patient. If a surgeon does not identify lymph node spread, the patient likely will not receive the proper treatment, which can be detrimental to their prognosis.

When resecting tumors after they have been located, the surgeon also aims to obtain clean margins surrounding the tumor, which are usually considered to be a 2 cm cancer-free zone in all directions of the tumor. In cases where the margins of the tumor are well defined, achieving clean margins is not particularly difficult. However, it is possible that the tumor, especially around the edges, is not well defined or has small cancerous projections that invade the surrounding normal tissue, which makes achieving clean margins particularly challenging.

For example, when viewing the tumor under white light, it may appear as if the surgeon has localized the tumor even though cancerous tissue remains in the patient's airway surrounding the resected tumor. If VGT-309 were not used, the surgeon may have left behind this cancerous tissue, or would have too aggressively resected the area to ensure suitable margins. However, using VGT-309, the surgeon can visualize any residual cancerous tissue and remove the entire tumor with clean margins, as verified by pathology, while preserving as much healthy tissue as possible.

VGT-309 and NIR therefore can help surgeons identify any potentially affected lymph nodes because lymph nodes containing cancer will be fluorescent under NIR.

Using existing surgical techniques, surgeons often struggle to locate and remove small tumors, which are often considered to be any tumors smaller than 2 cm. While this tumor may be clearly distinguishable on the patient's preoperative scans, it may be difficult for a surgeon to identify during a MIS procedure. For example, when viewing the surface of the lung under ordinary white light, the surgeon will find it difficult to locate the tumor. If this procedure were being performed without VGT-309 and NIR, the surgeon may be prompted to aggressively resect the tissue in this area or convert to an open procedure.

The methods described herein with respect to use of VGT-309 may also be carried out with other fluorescent molecular imaging agents that covalently bind to a target molecule that is present at larger amounts in solid tumors than in normal tissues effective to cause cancerous tissues to fluoresce under NIR light and facilitate localization of the cancerous tissues.

While the disclosure has been described with reference to a number of exemplary applications it will be understood by those skilled in the art that the disclosure is not limited to such disclosed embodiments. Rather, the disclosed applications can be modified to incorporate any number of variations, alterations, substitutions, or equivalent arrangements not described herein, but which are commensurate with the spirit and scope of the disclosure.

### EXAMPLES

This invention can be further understood with reference to the following non-limiting examples.

### Example 1. Localization of Tumor with VGT-309 and NIR Imaging

A patient was given 0.32 mg/kg of VGT-309 approximately 21 hours prior to surgery. A surgeon used NIR imaging to improve visualization and clearly show the location and extent of the tumor.

### Example 2. Localizing Undefined Tumors with VGT-309 and NIR Imaging and Resecting Localized Tumors with Clean Margins

A patient appeared to have a localized tumor, as identified by the preoperative scans, but the surgeon identified lymph node spread during resection with VGT-309. The patient's preoperative PET scan showed a large and easily identifiable tumor. Absent from these scans were any evidence of spread to other areas of the lungs or the lymph nodes. The patient was given 0.16 mg/kg of VGT-309 approximately 21 hours prior to surgery. Tissue was visualized under NIR to more easily locate the primary tumor during surgery. The surgeon also visually examined and biopsied the proximal lymph nodes, neither of which indicated the tumor had spread. But during the procedure, the surgeon observed unexpected fluorescence in a more distant lymph node. The surgeon was therefore able to perform a biopsy, which confirmed that the cancer had spread, and as a result re-staged the patient in real-time and performed a radical lymph node dissection. At the end of the procedure, the surgeon removed all traces of the cancer.

### Example 3. Identifying Metastatic Spread

A patient who, after radiation treatment of a primary tumor, experienced tumor regrowth but the patient's scans did not indicate metastatic spread. Here, the patient was given 0.32 mg/kg of VGT-309 approximately 18.5 hours prior to surgery, and the surgeon was able to quickly identify and remove the previously identified tumor. In addition to removing the primary tumor, the surgeon also was able to identify additional tumors using NIR, because the patient was given VGT-309 prior to surgery. When visualized under ordinary white light, the patient's lung tissue showed some signs of abnormality, but abnormalities are not always indicative of cancerous tissue. However, it was confirmed under NIR that this abnormal lung tissue was in fact cancerous.

### Example 4. Increasing Clinically Significant Events During Tumor Removal

The use of VGT-309 and NIR was shown to materially increase the likelihood of clinically significant events (CSEs) during removal procedures. To be clear, CSEs in this context are good in that they have been used a primary endpoint in clinical studies to show that imaging agents have the ability to change the decision making for the better during surgery. CSEs are one way to measure whether an imaging agent, such as VGT-309, adds a clinical benefit to the diagnosis and treatment process. In this context, CSEs generally fall into one of three categories: (1) localization of hard-to-find tumors, (2) identification of positive resection margins, and (3) identification of additional primary and metastatic lesions that were not previously detected using existing technologies. In studies, VGT-309 was shown to increase CSEs as compared to other, comparable imaging agents that are being developed. In a Phase 2 study of VGT-309 in Australia, one third of patients experienced a CSE, which is superior to the results from a Phase 2 study of a comparable imaging agent, where about one fourth of the patients experienced a CSE. VGT-309 therefore is increasing the frequency of CSE's, which indicates that VGT-309 is effective in helping surgeons (1) identify and localize the primary tumor, (2) resect the tumor with clean margins, and (3) identify additional unknown primary and metastatic lesions.

Some embodiments of the present disclosure can be described in view of one or more of the following:

Embodiment 1. A method comprising: navigating an instrument, via a minimally invasive route, into a patient to whom a molecular imaging agent has been intravenously administered to position the instrument in an area of a tissue abnormality; visualizing, via the instrument, tissue in the area under near-infrared (NIR) light, wherein the molecular imaging agent, as administered, is effective to cause abnormal tissue in the area to fluoresce under the NIR light and enable the fluorescing abnormal tissue to be localized within the area; and, optionally, then diagnosing and/or treating the localized fluorescing abnormal tissue.

Embodiment 2. The method of Embodiment 1, wherein the navigating via a minimally invasive route is an endoluminal procedure.

Embodiment 3. The method of Embodiment 1 or 2, wherein the area of the tissue abnormality is identified from one or more preoperative scans.

Embodiment 4. The method of any one of Embodiments 1 to 3, wherein the instrument comprises a biopsy needle or other biopsy tool; and the diagnosing and/or treating comprises collecting one or more biopsy samples from the localized fluorescing abnormal tissue.

Embodiment 5. The method of any one of Embodiments 1 to 4, wherein the diagnosing and/or treating comprises resection or destruction of the localized fluorescing abnormal tissue (e.g., cancerous tissue).

Embodiment 6. The method of Embodiment 5, wherein the destruction of the localized fluorescing abnormal tissue comprise (i) ablation (e.g., by means of heat, microwave, RF (radiofrequency) energy, laser, ultrasound, or histotripsy), and/or (ii) local administration of a drug (e.g., a chemotherapy agent, an antibody, an immuno-oncology agent, an oncolytic virus, a cell therapy) to the localized fluorescing abnormal tissue.

Embodiment 7. The method of any one of Embodiments 1 to 6, wherein the tissue abnormality comprises a tumor with poorly defined margins.

Embodiment 8. The method of any one of Embodiments 1 to 7, wherein the localized fluorescing abnormal tissue is in the patient's lungs.

Embodiment 9. The method of Embodiment 8, wherein the localized fluorescing abnormal tissue comprises a nodule external to a bronchus.

Embodiment 10. The method of Embodiment 8 or 9, wherein: the instrument comprises a flexible bronchoscope, and the navigating comprises guiding the flexible bronchoscope through the patient's airway.

Embodiment 11. The method of any one of Embodiments 1 to 7, wherein the localized fluorescing abnormal tissue is in the patient's colon.

Embodiment 12. The method of Embodiment 11, wherein the localized fluorescing abnormal tissue comprises a flat or depressed lesion.

Embodiment 13. The method of Embodiment 11 or 12, wherein: the instrument comprises an endoscope, and the navigating comprises guiding the endoscope through the patient's colon.

Embodiment 14. The method of any one of Embodiments 1 to 13, wherein an NIR-enabled confocal microscope is inserted into the fluorescing tissue to directly visualize live cells and their organization within the tissue abnormality.

Embodiment 15. The method of any one of Embodiments 1 to 14, which is done without a preoperative marking procedure.

Embodiment 16. The method of any one of Embodiments 1 to 15, wherein the molecular imaging agent is administered between 2 hours and 7 days, or from 2 hours to 4 days, e.g., between 72 hours and 96 hours, prior to the navigating.

Embodiment 17. The method of Embodiment 16, wherein the molecular imaging agent is administered between 12 hours and 36 hours prior to the navigating.

Embodiment 18. The method of any one of Embodiments 1 to 17, wherein the molecular imaging agent is administered to the patient at a dose from 0.01 mg/kg to 0.7 mg/kg.

Embodiment 19. The method of Embodiment 18, wherein the dose is from 0.015 mg/kg to 0.65 mg/kg, e.g., from 0.15 mg/kg to 0.65 mg/kg.

Embodiment 20. The method of Embodiment 18, wherein the dose is from 0.1 mg/kg to 0.6 mg/kg, e.g., from 0.3 mg/kg to 0.5 mg/kg.

Embodiment 21. The method of any one of Embodiments 1 to 20, wherein the molecular imaging agent is configured to covalently bind to a target molecule that is upregulated in solid tumors or present at larger amounts in solid tumors than in normal tissues.

Embodiment 22. The method of any one of Embodiments 1 to 21, wherein the molecular imaging agent is configured to bind to a cathepsin.

Embodiment 23. The method of any one of Embodiments 1 to 22, wherein the molecular imaging agent comprises VGT-309 (e.g., a pharmaceutically acceptable salt of VGT-309).

Embodiment 24. The method of any one of Embodiments 1 to 23, wherein the localized fluorescing abnormal tissue is malignant, or cancerous.

Embodiment 25. A method comprising: navigating an instrument, via an endoluminal route, into a patient to whom VGT-309 has been intravenously administered, to position the instrument in a target area; visualizing, via the instrument, the target area under near-infrared (NIR) light; and identifying in real time the location of abnormal tissue within the target area by the florescence of the abnormal tissue caused by the VGT-309 under the NIR light.

Embodiment 26. The method of Embodiment 25, wherein the VGT-309 is administered between 2 hours and 4 days, e.g., between 72 hours and 96 hours, prior to the navigating.

Embodiment 27. The method of Embodiment 26, wherein the VGT-309 is administered between 12 hours and 36 hours prior to the navigating.

Embodiment 28. The method of any one of Embodiments 25 to 28, wherein the VGT-309 is administered to the patient at a dose from 0.01 mg/kg to 0.7 mg/kg, e.g., from 0.1 mg/kg to 0.7 mg/kg, from 0.2 mg/kg to 0.6 mg/kg, or from 0.3 mg/kg to 0.5 mg/kg.

Embodiment 29. A method of performing a biopsy comprising: identifying a tissue abnormality on a patient's preoperative scans; intravenously administering a molecular imaging agent to the patient; navigating a biopsy needle to the location of the tissue abnormality; visualizing the tissue under near-infrared (NIR) light, wherein the molecular imaging agent causes abnormal tissue at the location to fluoresce under the NIR light; and collecting one or more biopsy samples from the fluorescing tissue, wherein the biopsy needle is navigated to the location via a minimally invasive route and the fluorescent tissue guides (i) the navigation of the biopsy needle and/or (ii) the collection of the one or more biopsy samples.

Embodiment 30. A method of removing or destroying a tumor in a patient, the method comprising: intravenously administering a molecular imaging agent to the patient; navigating a surgical instrument to a location of the tumor, as indicated by a preoperative scan and/or biopsy; visualizing tissue at the location under near-infrared (NIR) light, wherein the molecular imaging agent is effective to cause cancerous tissue in the tumor at the location to fluoresce under the NIR light; and then resecting or destroying the fluorescing tissue of the tumor, using the surgical instrument; wherein the surgical instrument is navigated to the location via a minimally invasive route and the fluorescent tissue guides (i) the navigation of the surgical instrument and/or (ii) the resecting or destroying.

Embodiment 31. The method of Embodiment 29 or 30, wherein the molecular imaging agent is administered between 2 hours and 4 days, e.g., between 72 hours and 96 hours, prior to the navigating.

Embodiment 32. The method of Embodiment 31, wherein the molecular imaging agent is administered between 12 hours and 36 hours prior to the navigating.

Embodiment 33. The method of any one of Embodiments 29 to 32, wherein the molecular imaging agent is administered to the patient at a dose from 0.01 mg/kg to 0.7 mg/kg, e.g., from 0.1 mg/kg to 0.7 mg/kg, from 0.2 mg/kg to 0.6 mg/kg, or from 0.3 mg/kg to 0.5 mg/kg.

Embodiment 34. The method of any one of Embodiments 29 to 33, wherein the molecular imaging agent is configured to covalently bind to a target molecule that is upregulated in solid tumors or present at larger amounts in solid tumors than in normal tissues.

Embodiment 35. The method of any one of Embodiments 29 to 34, wherein the molecular imaging agent is configured to bind to a cathepsin.

Embodiment 36. The method of any one of Embodiments 29 to 35, wherein the molecular imaging agent comprises VGT-309 (e.g., a pharmaceutically acceptable salt of VGT-309).

Embodiment 37. The method of any one of Embodiments 1 to 36, wherein the navigating of the instrument comprises use of a robotic or robotically-assisted system.

That which is claimed is:

1. A method comprising:
navigating an instrument, via a minimally invasive route, into a human patient to whom a molecular imaging agent has been intravenously administered, to position the instrument in an area of a tissue abnormality; and
visualizing, via the instrument, tissue in the area under near-infrared (NIR) light, wherein the molecular imaging agent, as administered, causes abnormal tissue in the area to fluoresce under the NIR light and enable the fluorescing abnormal tissue to be localized within the area,
wherein the molecular imaging agent comprises VGT-309 or a pharmaceutically acceptable salt thereof, and
wherein the molecular imaging agent is administered to the patient at a dose from 0.01 mg/kg to 0.7 mg/kg between 2 hours and 4 days prior to the navigating.

2. The method of claim 1, wherein the molecular imaging agent is administered between 72 hours and 96 hours prior to the navigating.

3. The method of claim 1, wherein the dose is from 0.015 mg/kg to 0.65 mg/kg.

4. The method of claim 1, wherein the dose is from 0.1 mg/kg to 0.4 mg/kg.

5. The method of claim 1, wherein the dose is from 0.015 mg/kg to 0.65 mg/kg and is administered between 72 hours and 96 hours prior to the navigating.

6. The method of claim 1, wherein the fluorescing abnormal tissue is in the patient's lungs.

7. The method of claim 6, wherein the fluorescing abnormal tissue comprises a nodule external to a bronchus.

8. The method of claim 6, wherein:
the instrument comprises a flexible bronchoscope, and
the navigating comprises guiding the flexible bronchoscope through the patient's airway.

9. The method of claim 1, further comprising diagnosing and/or treating the fluorescing abnormal tissue.

10. The method of claim 1, wherein the navigating, via a minimally invasive route, is an endoluminal procedure.

11. The method of claim 1, wherein the area of the tissue abnormality is identified from one or more preoperative scans.

12. The method of claim 1, wherein:
the instrument comprises a biopsy needle or other biopsy tool; and
the diagnosing and/or treating comprises collecting one or more biopsy samples from the fluorescing abnormal tissue.

13. The method of claim 1, wherein the diagnosing and/or treating comprises resection or destruction of the fluorescing abnormal tissue.

14. The method of claim 13, wherein the destruction of the fluorescing abnormal tissue comprise (i) ablation, and/or (ii) local administration of a drug to the fluorescing abnormal tissue.

15. The method of claim 1, which is done without a preoperative marking procedure.

16. The method of claim 1, wherein the navigating the instrument comprises use of a robotic or robotically-assisted system.

17. A method comprising:
navigating an instrument, via an endoluminal route, into a patient to whom VGT-309 or a pharmaceutically acceptable salt thereof has been intravenously administered, to position the instrument in a target area;
visualizing, via the instrument, the target area under near-infrared (NIR) light; and
identifying in real time the location of any abnormal or cancerous tissue within the target area by the florescence of the abnormal or cancerous tissue caused by the VGT-309 under the NIR light.

18. The method of claim 17, wherein the VGT-309 is administered between 2 hours and 4 days prior to the navigating.

19. The method of claim 17, wherein the VGT-309 is administered between 12 hours and 36 hours prior to the navigating.

20. The method of claim 17, wherein the VGT-309 is administered between 72 hours and 96 hours prior to the navigating.

21. The method of claim 17, wherein the VGT-309 is administered to the patient at a dose from 0.015 mg/kg to 0.65 mg/kg.

22. The method of claim 17, wherein the VGT-309 is administered to the patient at a dose from 0.015 mg/kg to 0.65 mg/kg between 48 hours and 96 hours prior to the navigating.

23. The method of claim 17, wherein the VGT-309 is administered to the patient at a dose from 0.1 mg/kg to 0.4 mg/kg between 48 hours and 96 hours prior to the navigating.

24. The method of claim 17, wherein the VGT-309 is administered to the patient at a dose from 0.015 mg/kg to 0.65 mg/kg between 72 hours and 96 hours prior to the navigating.

25. The method of claim 17, wherein the navigating the instrument comprises use of a robotic or robotically-assisted system.
